(19) United States
(12) Patent Application Publication
(10) Pub. No.: US 2020/0342342 A1
(43) Pub. Date: Oct. 29, 2020

(54) METHODS OF CREATING TRAIT PREDICTION MODELS AND METHODS OF PREDICTING TRAITS

(71) Applicant: IWATE MEDICAL UNIVERSITY, Iwate (JP)

(72) Inventor: Tsuyoshi HACHIYA, Iwate (JP)

(21) Appl. No.: 16/929,282

(22) Filed: Jul. 15, 2020

Related U.S. Application Data
(62) Division of application No. 15/529,636, filed on Aug. 3, 2017.

(30) Foreign Application Priority Data
Nov. 25, 2014 (JP) 2014-238252

(51) Int. Cl. G06N 7/00; G16B 5/00; G16B 40/00; G16H 10/60; G16B 20/00; G06F 17/16

(52) U.S. Cl. CPC G06N 7/005; G16B 5/00; G06F 17/16; G16H 10/60; G16B 20/00; G16B 40/00

(57) ABSTRACT

To provide methods of creating trait prediction models for predicting phenotypes of traits from single nucleotide polymorphism data and methods of predicting traits with which traits can be predicted with a high accuracy.

This is a method of creating a trait prediction model for predicting a phenotype of a multifactorial trait using data of a plurality of single nucleotide polymorphisms linked to a trait for each of a plurality of individuals of an organism: representing each of the plurality of single nucleotide polymorphisms as a matrix; classifying the plurality of single nucleotide polymorphisms into a plurality of categories based on their genetic architectures; calculating, for each of the categories, a genomic similarity matrix using the represented matrix and the number of the single nucleotide polymorphisms belonging to the category; and applying the genomic similarity matrix and a parameter of the genetic architecture to a linear mixed model.

### CROSS REFERENCE TO RELATED DOCUMENT

The present application claims the priority of Japanese Patent Application No. 2014-238252 filed Nov. 25, 2014, which is incorporated herein by reference.

### TECHNICAL FIELD

The present invention relates to methods of creating trait prediction models and methods of predicting traits.

### BACKGROUND ART

For phenotypic prediction using human genomic information, methods of predicting a phenotype using only a susceptibility polymorphism already identified have mainly been investigated, focusing on trait susceptibility polymorphisms (see, V. Lyssenko et al., N Engl J Med 2008 vol. 359 p. 2220-2232; S. Ripatthi et al., Lanet 2010 Vol. 376 p. 1393-1400; C. A. Ibrahim-Verbaas et al., Stroke 2014 vol. 45 p. 403-412). These methods enumerate several hundred polymorphisms related to traits and estimate a weight of each polymorphism; they are thus easy to be intuitively understood since effects of individual polymorphisms on traits can be expressed numerically.

The sole use of the susceptibility polymorphisms is, however, a disadvantage and the limit of this approach. This is because in almost all multifactorial traits, only a few of the susceptibility polymorphisms that are actually responsible have been identified. For example, it is estimated that about 80% of the variance in body height can be explained by genetic factors, but the variance explained by a known susceptibility polymorphism is only about 5%.

With this respect, non-patent literature document (D. Speed and D. J. Balding, Genome Research 2015 vol. 24 p. 1550-1557) discloses a method of predicting phenotypes using exhaustive (genome-wide) polymorphism information regardless of susceptibility polymorphisms. Specifically, a plurality of single nucleotide polymorphisms (SNPs) are divided into a plurality of categories, and a linear mixed model is applied thereto. The accuracy of prediction of the method is, however, still insufficient.

### SUMMARY OF INVENTION

### Technical Problem

An object of the present invention is to provide methods of creating trait prediction models for predicting phenotypes of traits from single nucleotide polymorphism data and methods of predicting traits with which traits can be predicted with a high accuracy.

### Solution to Problem

The present inventors have investigated a statistical processing method using exhaustive (i.e. genome-wide) polymorphism information regardless of susceptibility polymorphisms. Specifically, taking 27 qualitative traits including the body height and HbA1c value and 5 qualitative traits including diseases of diabetes and low HDL cholesterolemia as examples, the present inventors utilized a linear mixed model using about 1 million polymorphisms as genomic information and gender/age information as adjustment variables and trained the model about the traits to create a prediction model. The present inventors found that this prediction was highly correlated with measured values, and thus accomplished a method of predicting phenotypes from genomic information.

An aspect of the present invention is a method of creating a trait prediction model for predicting a phenotype of a multifactorial trait using data of a plurality of single nucleotide polymorphisms linked to a trait for each of a plurality of individuals of an organism, the method including the steps of: representing each of the plurality of single nucleotide polymorphisms as a matrix; classifying the plurality of single nucleotide polymorphisms into a plurality of categories based on their genetic architectures; calculating, for each of the categories, a genomic similarity matrix using the represented matrix and the number of the single nucleotide polymorphisms belonging to the category; and applying the genomic similarity matrix and a parameter of the genetic architecture to a linear mixed model. The genetic architecture may be an effect size and/or an allele frequency.

Another aspect of the present invention is a method of creating a trait prediction model for predicting a phenotype of a multifactorial trait using data of gender, age and a plurality of single nucleotide polymorphisms linked to a trait for each of a plurality of individuals of an organism, the method including the steps of: representing each of the plurality of single nucleotide polymorphisms as a matrix; representing the gender and/or age as a matrix; calculating a genomic similarity matrix using the represented matrix of the single nucleotide polymorphisms and the number of the single nucleotide polymorphisms; and applying the genomic similarity matrix and the matrix of the gender and/or age to a linear mixed model. The trait may be selected from the group consisting of the body height, body weight, systolic blood pressure, diastolic blood pressure, blood glucose, HbA1c, red blood cell number, hemoglobin, corpuscular volume, white blood cell number, platelet number, percentage of neutrophils, percentage of lymphocytes, percentage of monocytes, percentage of eosinophils, percentage of basophils, percentage of large unstained cells, AST (GOT), ALT (GPT), γ-GTP, total cholesterol, neutral fat, HDL cholesterol, LDL cholesterol, creatinine, urea nitrogen, uric acid, diabetes, hypertension, high LDL cholesterolemia, low HDL cholesterolemia, and hypertriglyceridemia.

A further aspect of the present invention is a method of predicting a trait of an individual of an organism from a plurality of single nucleotide polymorphism data in the individual of the organism, including the steps of: creating a prediction model using a set of training data according to the aforementioned method of creating a trait prediction model; determining a parameter and a hidden variable of a linear mixed model; and applying the plurality of single nucleotide polymorphism data of the individual of the organism to the prediction model.

A yet further aspect of the present invention is a program for predicting a trait of an individual of an organism from a plurality of single nucleotide polymorphism data in the individual of the organism, by which the computer is caused to execute the aforementioned method of predicting a trait. An aspect of the present invention may be a computer readable recording medium in which the present program has been recorded.

A further aspect of the present invention is a trait prediction system for predicting a trait of an individual of an organism from a plurality of single nucleotide polymorphism data, including: (i) an input device for inputting a plurality of single nucleotide polymorphism data of the individual of the organism; (ii) a computer that executes the above program using data that has been input, and (iii) an output device for outputting the result obtained in (ii).

### BRIEF DESCRIPTION OF DRAWINGS

FIG. 1 represents a diagram showing estimated contribution ratios (with Qₑₛ=50 and Q_{RAF}=1) obtained by a genetic architecture division method, focusing on HbA1c values and body heights, in an example of the present invention.

FIG. 2 represents a diagram showing estimated contribution ratios (with Qₑₛ=1 and Q_{RAF}=30) obtained by a genetic architecture division method, focusing on HbA1c values and body heights in an example of the present invention.

FIG. 3 represents a list of traits used in examples of the present invention.

FIG. 4 represents a diagram showing results of accuracy evaluation for 27 quantitative traits in an example of the present invention. The following three cases were compared: (1) only the single nucleotide polymorphism information was used and Qₑₛ=1 and Q_{RAF}=1 (without the genetic architecture division); (2) only the gender/age information was used; and (3) both the single nucleotide polymorphism information and the gender/age information were used and Qₑₛ=1 and Q_{RAF}=1 (without the genetic architecture division; the examples of the present invention). A coefficient of determination R² between measured and predicted values (i.e., a squared correlation coefficient) was used as an evaluation measure and the evaluation was performed using a 2-fold cross validation method.

FIG. 5 represents a diagram showing results of accuracy evaluation for 5 qualitative traits in an example of the present invention. The following three cases were compared: (1) only the single nucleotide polymorphism information was used and Qₑₛ=1 and Q_{RAF}=1 (without the genetic architecture division); (2) only the gender/age information was used; and (3) both the single nucleotide polymorphism information and the gender/age information were used and Qₑₛ=1 and Q_{RAF}=1 (without the genetic architecture division; the examples of the present invention). AUC was used as an evaluation measure and the evaluation was performed using a 2-fold cross validation method.

FIG. 6 represents a diagram showing results of accuracy evaluation for 27 quantitative traits with sufficient amount of samples in an example of the present invention. The following four methods were compared: (1) only the single nucleotide polymorphism information was used and Qₑₛ=1 and Q_{RAF}=1 (without the genetic architecture division); (2) only the gender/age information was used; (3) both the single nucleotide polymorphism information and the gender/age information were used and Qₑₛ=1 and Q_{RAF}=1 (without the genetic architecture division; the examples of the present invention); and (4) both the single nucleotide polymorphism information and the gender/age information were used and Qₑₛ=10 and Q_{RAF}=1 (with the genetic architecture division; the examples of the present invention). A coefficient of determination R² between measured and predicted values (i.e., a squared correlation coefficient) was used as an evaluation measure and the evaluation was performed using a 2-fold cross validation method.

FIG. 7 represents a diagram showing results of accuracy evaluation for 5 qualitative traits with sufficient amount of samples in an example of the present invention. The following four methods were compared: (1) only the single nucleotide polymorphism information was used and Qₑₛ=1 and Q_{RAF}=1 (without the genetic architecture division); (2) only the gender/age information was used; (3) both the single nucleotide polymorphism information and the gender/age information were used and Qₑₛ=1 and Q_{RAF}=1 (without the genetic architecture division; the examples of the present invention); and (4) both the single nucleotide polymorphism information and the gender/age information were used and Qₑₛ=10 and Q_{RAF}=1 (with the genetic architecture division; the examples of the present invention). AUC was used as an evaluation measure and the evaluation was performed using a 2-fold cross validation method.

### DESCRIPTION OF EMBODIMENTS

The objects, features, advantages, and ideas of the present invention are apparent to those skilled in the art from the description of this specification. Furthermore, those skilled in the art can easily reproduce the present invention from the description herein. The embodiments and specific examples described below represent preferable embodiments of the present invention, which are given for the purpose of illustration or explanation. The present invention is not limited thereto. It is obvious to those skilled in the art that various changes and modifications may be made according to the description of the present specification within the spirit and scope of the present invention disclosed herein.

A method of creating a trait prediction model according to the present invention is a method of creating a trait prediction model for predicting a phenotype of a multifactorial trait using data of a plurality of single nucleotide polymorphisms linked to a trait for each of a plurality of individuals of an organism, the method including the steps of: representing each of the plurality of single nucleotide polymorphisms as a matrix; classifying the plurality of single nucleotide polymorphisms into a plurality of categories based on their genetic architectures; calculating, for each of the categories, a genomic similarity matrix using the represented matrix of the single nucleotide polymorphisms and the number of the single nucleotide polymorphisms belonging to each category; and applying the genomic similarity matrix and a parameter of the genetic architecture to a linear mixed model; or a method of creating a trait prediction model for predicting a phenotype of a multifactorial trait using data of gender, age and a plurality of single nucleotide polymorphisms linked to a trait for each of a plurality of individuals of an organism, the method including the steps of: representing each of the plurality of single nucleotide polymorphisms as a matrix; representing the gender and/or age as a matrix; calculating a genomic similarity matrix using the represented matrix of the single nucleotide polymorphisms and the number of the single nucleotide polymorphisms; and applying the genomic similarity matrix and the matrix of the gender and/or age to a linear mixed model.

The single nucleotide polymorphisms contained in the single nucleotide polymorphism data used here are not particularly limited and may or may not be a susceptibility polymorphism on a target trait. The number and type of the single nucleotide polymorphisms to be used are also not particularly limited, but it is preferable to encompass all single nucleotide polymorphisms that occur at a frequency of at least 1% in a population of individuals of a target organism.

The target organism is not particularly limited, and it may be a plant or an animal, but the target organism is preferably a vertebrate, more preferably a mammal, and most preferably human. The target trait is not particularly limited as long as it is a multifactorial trait, and for example, in the case of human, examples of the traits include indexes relating to the body such as the body height, body weight and BMI; blood test values such as blood pressure (i.e., systolic blood pressure and/or diastolic blood pressure), HbA1c, red blood cell number, hemoglobin, corpuscular volume, white blood cell number, platelet number, percentage of neutrophils, percentage of lymphocytes, percentage of monocytes, percentage of eosinophils, percentage of basophils, percentage of large unstained cells, percentage of nucleated red blood cells, AST (GOT), ALT (GPT), γ-GTP, total cholesterol, neutral fat, HDL cholesterol, LDL cholesterol, creatinine, urea nitrogen, estimated glomerular filtration rate, and uric acid; abilities such as memory, understanding, intelligence index, and exercise skill; and susceptibility to diseases such as lifestyle related diseases including obesity, diabetes, hypertension, and cardiovascular disease, cancer, and immunity diseases including allergy and autoimmune diseases.

By using the method of creating a prediction model of the present invention, it is possible to predict a trait of an individual of an organism from a plurality of single nucleotide polymorphism data. More specifically, a trait prediction model is created and parameters and hidden variables of the linear mixed model are determined using a set of training data according to the method of creating a trait prediction model of the present invention; and then a plurality of single nucleotide polymorphism data are applied to the trait prediction model, thereby it is possible to predict traits of the individual of the organism.

Hereinafter, methods of creating a prediction model and methods of predicting traits of the present invention will be described in detail and specifically with referring to examples, but the present invention is not limited to these embodiments or examples.

### (1) Matrix Representation Of Gender/Age Information

Given that gender and age data have already been obtained for N human individuals, a process of representing these data as an N-by-6 matrix X is described. Each row vector of the matrix X represents the gender/age information of the corresponding individual. An element in the i-th row and j-th column of the matrix X is herein denoted as X(i,j). Age is treated as categorical data, but the number of categories is not particularly limited. Here, described is an example where the following five categories are used: age 39 or younger, age 40 to 49, age 50 to 59, age 60 to 69, and age 70 or over.

The gender information is arranged at the first column of the matrix X. When the i-th human individual is given a gender designation “M” for male and “F” for female, an element X(i,1) is defined by:

$$X ( i , 1 ) = \{ \begin{matrix}0 & for {}_{ }^{″}{F}{}_{ }^{″} \\ 1 & for {}_{ }^{″}{M}{}_{ }^{″}\end{matrix} .$$

The age information is arranged at the columns 2 to 6 of the matrix X. When the age of the i-th human individual is ageᵢ, elements X(i,2), X(i,3), X(i,4), X(i,5), and X(i,6) are defined by:

$$X ( i , 2 ) = \{ \begin{matrix}1 & {age}_{i} \leq 39 \\ 0 & otherwise\end{matrix} \\ X ( i , 3 ) = \{ \begin{matrix}1 & 40 \leq {age}_{i} \leq 49 \\ 0 & otherwise\end{matrix} \\ X ( i , 4 ) = \{ \begin{matrix}1 & 50 \leq {age}_{i} \leq 59 \\ 0 & otherwise\end{matrix} \\ X ( i , 5 ) = \{ \begin{matrix}1 & 60 \leq {age}_{i} \leq 69 \\ 0 & otherwise\end{matrix} \\ X ( i , 6 ) = \{ \begin{matrix}1 & 70 \leq {age}_{i} \\ 0 & otherwise .\end{matrix}$$

### (2) Matrix Representation Of Genomic Information

Given that p single nucleotide polymorphism (SNP) data have already been obtained for N human individuals, a process of representing these data as an N-by-p matrix W (where N and p are each an integer of 1 or larger) is described. Each row vector of the matrix W represents a polymorphism profile in the corresponding individual and each column vector of the matrix W represents a vector indicating differences between or among individuals for a certain polymorphism site.

The j-th polymorphism of the i-th human individual has two alleles. An individual with both alleles identical to the human representative sequence is denoted as “AA”, a human with only one allele identical to the human representative sequence is denoted as “AB”, and a human with both alleles not identical to the human representative sequence is denoted as “BB”. The element in the i-th row and j-th column of the matrix W is denoted as W(i,j). The allele frequency of the j-th polymorphism is denoted as fⱼ. With these denotations, an element W(i,j) is defined by:

$$W ( {i}_{J} ) = \{ \begin{matrix}\frac{- 2 {f}_{j}}{\sqrt{2 {f}_{j} ( 1 - {f}_{j} )}} & for {}_{ }^{″}{AA}{}_{ }^{″} \\ \frac{1 - 2 {f}_{j}}{\sqrt{2 {f}_{j} ( 1 - {f}_{j} )}} & for {}_{ }^{″}{AB}{}_{ }^{″} \\ \frac{2 - 2 {f}_{j}}{\sqrt{2 {f}_{j} ( 1 - {f}_{j} )}} & for {}_{ }^{″}{BB}{}_{ }^{″}\end{matrix} .$$

The representative sequence herein is a sequence having nucleotides determined for respective polymorphisms, but it may be, for example, a publicly-available sequence that has been obtained in a genome project. (3) Classification of SNPs based on genetic architectures

A way of classifying p SNPs into multiple categories based on their genetic architectures is described below. Specific parameters of genetic architecture include an effect size, which is a parameter of the strength of the relationship with a trait, and an allele frequency, which represents the frequency of SNPs in a human population. Representative specific examples of the effect size include relative risk, odds ratio, coefficient of determination, and regression coefficient. Examples of the allele frequency include risk allele frequency (RAF) and minor allele frequency (MAF). Although the parameters describing the genetic architecture used in the method of the present invention are not specifically limited, a classification process with the regression coefficient and RAF is shown as an example.

### (4) Division Procedure (1): Calculation Of Qₑₛ Quantiles For Effect Sizes

For a positive integer Qₑₛ, (Qₑₛ−1) values dividing the distribution into Qₑₛ equal parts are calculated. A specific method of calculating quantiles is shown below, but the method of calculating the quantiles is not limited thereto. When the data obtained by sorting the effect sizes of the SNPs in ascending order is es₁≤es₂≤. . . ≤esₚ, the i-th Qₑₛ-quantile Qₑₛ⁽ⁱ⁾(1≤i≤Qₑₛ−1) is given by:

$${m}_{i} = \frac{i \times p}{{Q}_{es}}$$

$${m}_{i}^{L} = ⌊ {m}_{i} ⌋$$

$${m}_{i}^{H} = ⌈ {m}_{i} ⌉$$

$${Q}_{es}^{( i )} = \frac{{es}_{{m}_{i}^{L}} + {es}_{{m}_{i}^{H}}}{2} ,$$

where └mᵢ┘ and ┌mᵢ┐ are values obtained by rounding down and up the fractional part of mᵢ, respectively. For the sake of convenience, Qₑₛ⁽⁰⁾ and Qₑₛ^{(Q}^{ₑₛ}⁾ defined by:

Qₑₛ⁽⁰⁾=es₁

Qₑₛ^{(Q}^{ₑₛ}⁾=esₚ

### (5) Division Procedure (2): Calculation Of Q_{RAF} Quantiles For RAF

For a positive integer Q_{RAF}, (Q_{RAF}−1) values dividing the distribution into Q_{RAF} equal parts are computed. A specific method of calculating quantiles is shown below, but the method of calculating the quantiles is not limited thereto. When the data obtained by sorting RAFs of the SNPs in ascending order is RAF₁≤RAF₂≤ . . . ≤RAFₚ, the j-th Q_{RAF}-quantile Q_{RAF}^{(j)}(1≤j≤Q_{RAF}−1) is given by:

$${m}_{j} = \frac{j \times p}{{Q}_{RAF}}$$

$${m}_{j}^{L} = ⌊ {m}_{j} ⌋$$

$${m}_{j}^{H} = ⌈ {m}_{j} ⌉$$

$${Q}_{RAF}^{( j )} = \frac{{RAF}_{{m}_{j}^{L}} + {RAF}_{{m}_{j}^{H}}}{2} ,$$

where └mⱼ┘ and ┌mⱼ┐ are values obtained by rounding down and up the fractional part of mⱼ, respectively. For the sake of convenience, Q_{RAF}⁽⁰⁾ and Q_{RAF}^{(Q}^{_{RAF}}⁾ are defined by:

Q_{RAF}⁽⁰⁾=RAF₁

Q_{RAF}^{(Q}^{_{RAF}}⁾=RAFₚ

### (6) Classification of SNPs

The p SNPs are classified into Qₑₛ-by-Q_{RAF} categories using the results of Qₑₛ⁽ⁱ⁾(0≤i≤Qₑₛ) and Q_{RAF}-quantiles Q_{RAF}^{(j)}(0≤j≤Q_{RAF}) calculated by the aforementioned process. When the effect size and RAF of the k-th SNP (1≤k≤p) is esₖ and RAFₖ, respectively, a category catₖ of the k-th SNP is defined by:

catₖ=(i^{k}, j^{k})

s.t.Qₑₛ⁽ⁱ^{^{k}}⁻¹⁾≤esₖ≤Qₑₛ⁽ⁱ^{^{k}}⁻¹⁾, Q_{RAF}^{(j}^{^{k}}⁻¹⁾≤RAFₖ≤Q_{RAF}^{(j}^{^{k}}⁻¹⁾

### (7) Estimation Of Parameters Of Genetic Architecture

Parameters of genetic architecture such as the effect size and RAF can be estimated by association analysis of polymorphisms with traits. For the analysis of association between of polymorphisms and traits, a program available to the public can be used, and for example, PLINK or GCTA available on the Internet may be used.

### (8) Calculation Of Genomic Similarity Matrix

The “genomic similarity matrix” refers to an N-by-N matrix representing similarities between individuals based on genomic information. Here, the genomic similarity matrix is calculated for each of the Qₑₛ-by-Q_{RAF} categories. A typical equation for calculating a genomic similarity matrix A is shown below, but equations for calculating genomic similarity matrices are not limited thereto:

$${A}^{( i , j )} = \frac{1}{{p}^{( i , j )}} {W}^{( i , j )} {W}^{( i , j ) ′} ,$$

where A^{(i,j)} is a genomic similarity matrix (N by N dimensions) for the category (i,j), p^{(i,j)} is the number of SNPs belonging to the category (i,j), W^{(i,j)} is a submatrix (N by p^{(i,j)} dimensions) obtained by taking a column vector or vectors of SNPs belonging to the category (i,j) from the matrix W, and W^{(i,j)}′ is a transpose of the submatrix W^{(i,j)}.

### (9) Use Of Linear Mixed Models

### (9-1) Use Of Genetic Architectures

As a prediction model using genomic information, a linear mixed model is given by:

$$y = {μ1}_{N} + g + ɛ$$

$$g = {\sum }_{i , j} {g}^{( i , j )}$$

$${g}^{( i , j )} ∼ N ( 0 , {σ}_{g}^{{2}^{( i , j )}} {A}^{( i , j )} )$$

$$ɛ ∼ N ( 0 , {σ}_{e}^{2} I ) ,$$

where y is a vector (N dimension) of traits, μ is a mean value of traits, 1_{N} is a column vector (N dimension) of which elements are all 1, g is a vector (N dimension) of genetic contributions to a trait, ε is a residual vector (N dimension), g^{(i,j)} is a vector (N dimension) of contributions of SNPs belonging to the category (i,j) to a trait, A^{(i,j)} is a genomic similarity matrix (N by N dimensions) for the category (i,j), I is an identity matrix (N by N dimensions), N(0,σ_{g}^{2(i,j)}A^{(i,j)}) represents a multivariate normal distribution (with mean vector 0 and variance-covariance structure σ_{g}^{2(i,j)}A^{(i,j)}), and N(0,σₑ²I) represents a multivariate normal distribution (with mean vector 0 and variance-covariance structure σₑ²I).

### (9-2) With Gender/Age Information

As a prediction model using genomic information and gender/age information, a linear mixed model is given by:

y=μ1_{N}+Xβ+g+ε

g˜N(0,σ_{g}²A)

ε˜N(0,σₑ²I)

where y is a vector (N dimension) of traits, μ is a mean value of traits, 1_{N} is a column vector (N dimension) of which elements are all 1, X is a matrix (N by 6 dimensions) containing the gender/age information, , β is a weight for gender or age variables (6 dimension), g is a vector (N dimension) of genetic contributions to a trait, ε is a residual vector (N dimension), A is a genomic similarity matrix (N by N dimensions) when Qₑₛ=1 and Q_{RAF}=1, I is an identity matrix (N by N dimensions), N(0,σ_{g}²A) represents a multivariate normal distribution (with mean vector 0 and variance-covariance structure σ_{g}²A), and N(0,σₑ²I) represents a multivariate normal distribution (with mean vector 0 and variance-covariance structure σₑ²I).

### (9-3) With Genetic Architectures And Gender/Age Information

As a prediction model using genomic information and gender/age information, a linear mixed model is given by:

$$y = {μ1}_{N} + X β + g + ɛ$$

$$g = {\sum }_{i , j} {g}^{( i , j )} \\ {g}^{( i , j )} ∼ N ( 0 , {σ}_{g}^{{2}^{( i , j )}} {A}^{( i , j )} ) \\ ɛ ∼ N ( 0 , {σ}_{e}^{2} I ) ,$$

where y is a vector (N dimension) of traits, μ is a mean value of traits, 1_{N} is a column vector (N dimension) of which elements are all 1, X is a matrix (N by 6 dimensions) containing the gender/age information, , β is a weight for gender or age variables (6 dimension), g is a vector (N dimension) of genetic contributions to a trait, ε is an residual vector (N dimension), g^{(i,j)} is a vector (N dimension) of contributions of SNPs belonging to the category (i,j) to a trait, A^{(i,j)} is a genomic similarity matrix (N by N dimensions) for the category (i,j), I is an identity matrix (N by N dimensions), N(0,σ_{g}^{2(i,j)}A^{(i,j)}) represents a multivariate normal distribution (with mean vector 0 and variance-covariance structure σ_{g}^{2(i,j)}A^{(i,j)}), and N(0,σₑ²I) represents a multivariate normal distribution (with mean vector 0 and variance-covariance structure σₑ²I).

### (10) Estimation Of Parameters In Linear Mixed Models

Parameters (μ, β, σ_{g}^{2(i,j)}, σₑ²) in linear mixed models can be estimated using the restricted maximum likelihood (REML) approach. For REML, a commonly available program can be used, and GCTA which can be downloaded free of charge from the Internet or a commercial program ASReml may be used. Average Information REML, Fisher-scoring REML, and EM can be used for estimation of parameters in the GCTA and Average Information REML can be used for estimation of parameters in the ASReml. Hereinafter, the estimated parameters are denoted as \tilde{μ}, \hat{β}, [symbol] ^{(i,j)}, and [symbol] .

### (11) Estimation Of Contribution Ratio

A contribution ratio V_{G}^{(i,j)}/V_{P} for the SNPs belonging to the category (i,j) is defined by the following equation using the parameters ( [symbol] ^{(i,j)}, [symbol] ) estimated by REML:

$${V}_{G}^{( i , j )} \text{/} {V}_{P} = \frac{{( i , j )}^{}}{{( i , j )}^{} + {σ}_{e}^{2}} .$$

The total contribution ratio V_{G}/V_{P} for all SNPs is defined by:

$${V}_{G} \text{/} {V}_{P} = {\sum }_{i , j} {V}_{G}^{( i , j )} \text{/} {V}_{P} .$$

### (12) Prediction Of Contributions By Genetic Factors

Hidden variables (g, g^{(i,j)}, ε) of the linear mixed model are not included in the REML likelihood function and thus cannot be estimated, but they can be predicted by:

$${\hat{g}}^{( i , j )} = {( i , j )}^{} {A}^{( i , j )} Py$$

$$\hat{g} = {\sum }_{i , j} {\hat{g}}^{( i , j )}$$

$$\hat{ϵ} = y - \hat{g} ,$$

where P is an N-by-N matrix given by P=V⁻¹−V⁻¹\dot{X}(\dot{X}′V⁻¹\dot{X})⁻¹\dot{X}′V⁻¹, V is an N-by-N matrix given by V=Σ_{i,j} [symbol] ^{(i,j)}A^{(i,j)}+ [symbol] I, y is a vector (N dimension) of traits, and \dot{X} is an N-by-7 matrix given by \dot{X}=(1_{N},X). Hereinafter, the predicted hidden variables are denoted as ĝ, ĝ^{(i,j)}, and \hat{ϵ}.

### (13) Trait Prediction

When the estimated parameters (\hat{μ}ₜ, \hat{β}ₜ, [symbol] ₜ^{(i,j)}, [symbol] ₜ) and predicted hidden variables (ĝₜ^{(i,j)}, \hat{ϵ}ₜ) have been obtained using the aforementioned method from a set of training data (yₜ, Xₜ, Wₜ) for Nₜ individuals with all of the genomic information, gender/age information, and phenotypic information and genomic information (W_{ν}) and gender/age information (X_{ν}) for N_{ν} individuals to be predicted have been obtained but phenotypic information (y_{ν}) is unknown, a predicted value ŷ_{ν} (N dimension) of the unknown phenotypic information can be given by:

$${\hat{u}}_{t}^{( i , j )} = \frac{1}{N} {W}_{t}^{( i , j ) ′} {A}_{t}^{{( i , j )}^{- 1}} {\hat{g}}_{t}^{( i , j )} \\ {\hat{y}}_{v} = {\hat{μ}}_{t} {1}_{{N}_{v}} + {X}_{v} {\hat{β}}_{t} + {Σ}_{i , j} {W}_{v}^{( i , j )} {\hat{u}}_{t}^{( i , j )} \qquad ( 1 )$$

where Wₜ^{(i,j)} is a submatrix (Nₜ by p^{(i,j)} dimensions) obtained by taking a column vector or vectors of SNPs belonging to the category (i,j) from the matrix Wₜ, A^{(i,j)} is a genomic similarity matrix (Nₜ by Nₜ dimensions) calculated from Wₜ^{(i,j)}, ĝₜ^{(i,j)} is an predicted hidden variable (Nₜ dimension) calculated from a set of training data, \hat{μ}ₜ is a mean value of traits, 1_{N}_{_{ν}} is a column vector (N_{ν} dimension) of which elements are all 1, \hat{μ}ₜ^{(i,j)} is a weight vector (p^{(i,j)} dimension) for each SNP belonging to the category (i,j) calculated from a set of training data, and W_{ν}^{(i,j)} is a submatrix (N_{ν} by p^{(i,j)} dimensions) obtained by taking a column vector or vectors of SNPs belonging to the category (i,j) from a genomic information matrix W_{ν} for a set of data to be predicted.

As a special example of Equation (1), the following Equations (2) and (3) can be considered:

ŷ_{ν}=\hat{μ}ₜ1_{N}_{_{ν}}+X_{ν}\hat{β}ₜ (2)

ŷ_{ν}=\hat{μ}ₜ1_{N}_{_{ν}}+Σ_{i,j}W_{ν}^{(i,j)}ûₜ^{(i,j)} (3).

Equation (2) represents a equation for predicting traits using only the gender/age information, and Equation (3) represents a equation for predicting traits using only the genomic information. Furthermore, when Qₑₛ=1 and Q_{RAF}=1, then the following Equations (4) and (5) can be considered as special cases of Equations (1) and (3), respectively:

ŷ_{ν}=\hat{μ}ₜ1_{N}_{_{ν}}+X_{ν}\hat{β}ₜ+W_{ν}^{(1,1)}ûₜ^{(1,1)} (4)

ŷ_{ν}=\hat{μ}ₜ1_{N}_{_{ν}}+W_{ν}^{(1,1)}ûₜ^{(1,1)} (5).

Equation (1) is designated as a “genetic architecture division+gender/age adjustment method,” Equation (2) is designated as a “gender/age adjustment method,” Equation (3) is designated as a “genetic architecture division method,” Equation (4) is designated as a “genetic architecture non-division+gender/age adjustment method,” and Equation (5) is designated as a “genetic architecture non-division method.”

### (14) Trait Prediction System

In order to automate the aforementioned methods of predicting traits, they can be programmed so that they can be executed by a computer. A program thus created is also within the scope of the present invention.

Furthermore, a trait prediction system can be provided which has, in addition to the computer for executing the program, an input device for inputting information such as single nucleotide polymorphism, gender, and age and an output device for outputting results obtained by the execution of the program.

### EXAMPLES

Single nucleotide polymorphism information of the examples described below was measured using HumanOmniExpressExome chip (Illumina).

### Example 1

### Method

In this example, body heights were focused as an example of a multifactorial quantitative trait. Single nucleotide polymorphism data and gender/age information collected from 4,992 individuals from April 2015 to March 2016 by the Tohoku Medical Megabank Project were used and trait prediction models were made by the method of creating a trait prediction model of the present invention (using the aforementioned (9-2) with gender/age information) to estimate heritability. Heritability was also estimated as controls for cases where no gender/age information was used and compared with those in the cases where the information was used.

Next, the accuracy of prediction by the trait prediction model was evaluated for each of the cases where (1) only the gender/age information was used; (2) only the single nucleotide polymorphism information was used; and (3) both were used (i.e., the examples of the present invention), using a 2-fold cross validation method. The coefficient of determination R² (i.e., a squared correlation coefficient) between the measured value and the predicted value was used as an evaluation measure.

### Estimation Method Of Heritability

When Qₑₛ=1 and Q_{RAF}=1, the proportion of trait variance explained by genetic factors is referred to as heritability h². A heritability [symbol] is calculated by the following equation using the parameters ( [symbol] ^{(1,1)}, [symbol] ) estimated by REML:

$$\hat{{h}^{2}} = \frac{{\hat{{σ}_{g}^{2}}}^{( 1 , 1 )}}{{\hat{{σ}_{g}^{2}}}^{( 1 , 1 )} + \hat{{σ}_{e}^{2}}} .$$

### Results

The heritability obtained without using the gender/age information was 40.67% whereas the heritability obtained with using the gender/age information was 82.29%. The heritability was significantly increased when the gender/age information was used as compared with the case without using the gender/age information. It was found that a part of the variance of the body height can be accounted for by the gender and age.

The accuracies of prediction (R²) were evaluated for the three cases (1) to (3) using the 2-fold cross validation method (mean±standard deviation), which were (1) 56.89±1.36%, (2) 1.45±0.26%, and (3) 59.63±1.24%, respectively. When both of the gender/age information and the genome information were used, the accuracy of prediction increased as compared with the case where only the gender/age information was used and the case where only the genome information was used.

### Example 2

### Method

In this example, a disease of diabetes was focused as an example of a multifactorial quantitative trait. Single nucleotide polymorphism data and gender/age information collected from 4,992 individuals from April 2015 to March 2016 by the Tohoku Medical Megabank Project were used and trait prediction models were made by the method of creating a trait prediction model of the present invention (using the aforementioned (9-2) with gender/age information). According to the results of an HbA1c test, an individual was assumed to suffer from diabetes when the level was 6.5 or higher, and assumed not to suffer from diabetes when the level was lower than 6.5. The accuracy of prediction by the trait prediction model was evaluated for each of the cases where (1) only the gender/age information was used; (2) only the single nucleotide polymorphism information was used; and (3) both were performed (i.e., the examples of the present invention), using a 2-fold cross validation method. AUC was used as an evaluation measure.

### Results

The accuracies of prediction were (1) 61.39±1.56%, (2) 55.76±0.28%, and (3) 62.98±0.61%. When both of the gender/age information and the genome information were used, the accuracy of prediction increased as compared with the case where only the gender/age information was used and the case where only the genome information was used.

### Example 3

### Method

In this example, HbA1c levels and body heights were focused as examples of a multifactorial quantitative trait. Single nucleotide polymorphism data collected from 4,992 individuals from April 2015 to March 2016 by the Tohoku Medical Megabank Project were used to estimate contribution ratios by the genetic architecture division method. Estimation was performed for two cases: (1) when Qₑₛ=50 and Q_{RAF}=1, and (2) when Qₑₛ=1 and Q_{RAF}=30.

### Results

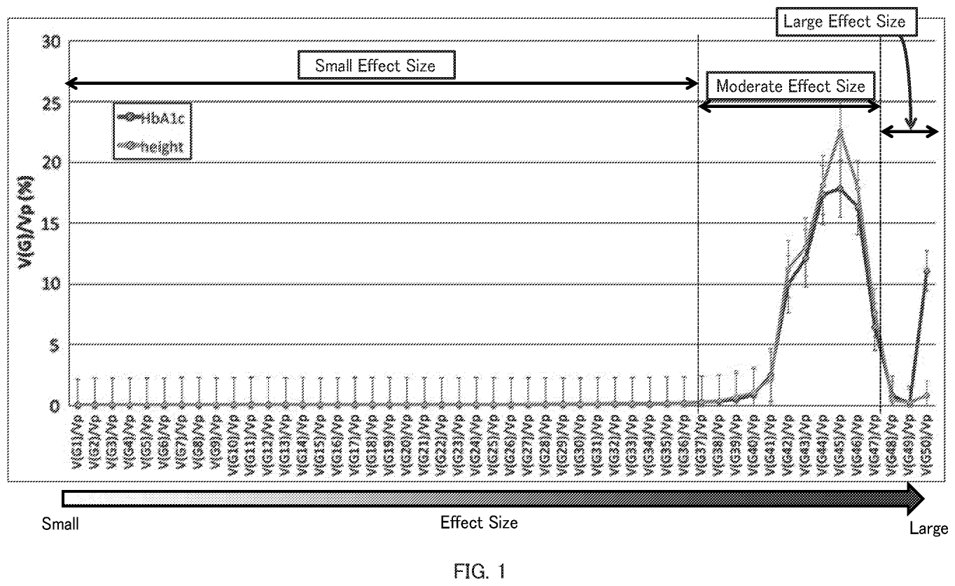

(1) FIG. 1 shows estimated contribution ratios with Qₑₛ=50 and Q_{RAF}=1. It was estimated that the contribution ratios for single nucleotide polymorphisms with moderate effect sizes are larger and the contribution ratios for single nucleotide polymorphisms with small effect sizes are extremely small both in the case using the HbA1c levels and the case using the body heights. It was also estimated that the contributions of the single nucleotide polymorphisms with larger effect sizes are large in the case using the HbA1c levels, but the contributions of the single nucleotide polymorphisms with large effect sizes are limited in the case using the body heights.

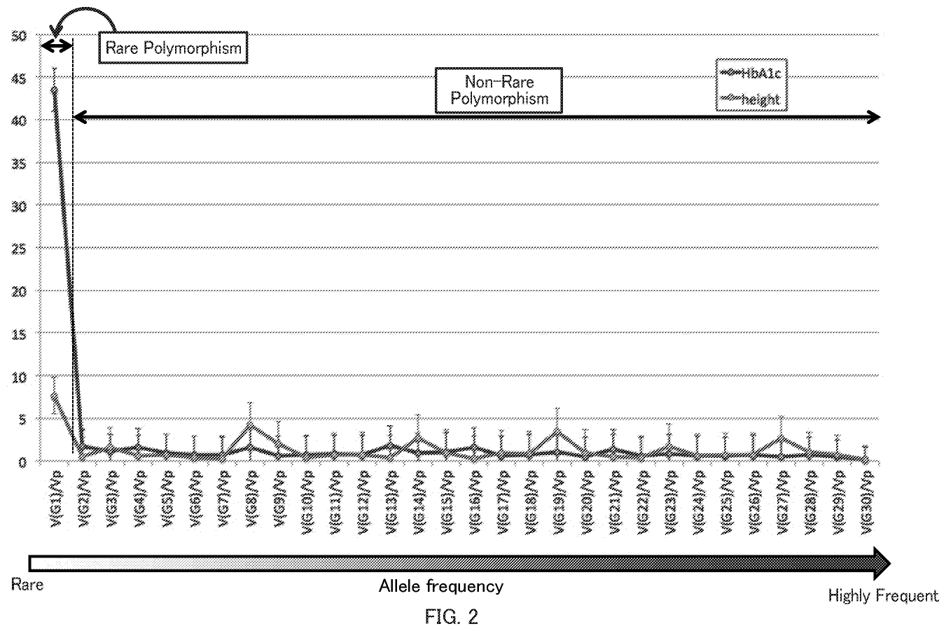

(2) FIG. 2 shows estimated contribution ratios with Qₑₛ=1 and Q_{RAF}=30. It was estimated that the contribution ratios for single nucleotide polymorphisms which are not rare are limited and the contribution ratios for single nucleotide polymorphisms which are rare are extremely high in the case using the HbA1c levels. It was also estimated that the contributions of the single nucleotide polymorphisms which are rare are not small but the contributions of the single nucleotide polymorphisms which are not rare are also not small in the case using the body heights.

### Example 4

### Method

In order to show that genetic architecture division method can improve the accuracy of trait prediction when trained with sufficient amount of samples, single nucleotide polymorphism data and HbA1c levels collected from 4,992 individuals from April 2015 to March 2016 by the Tohoku Medical Megabank Project were used. Estimation of effect sizes and allele frequencies as well as estimation of linear mixed models were performed using a set of verification data. Prediction of contribution ratio by genetic factors and calculation of weights to single nucleotide polymorphisms were performed using a set of training data. The accuracy of prediction was verified using a set of verification data. It is thus possible to evaluate the accuracy of prediction for cases where the sample size is sufficiently large.

The accuracies of prediction by the trait prediction models were evaluated for each of the cases with (1) Qₑₛ=1 and Q_{RAF}=1 (without the genetic architecture division) and (2) Qₑₛ=10 and Q_{RAF}=1 (with the genetic architecture division; the examples of the present invention), using the 2-fold cross validation method. The coefficient of determination R² (i.e., a squared correlation coefficient) between the measured value and the predicted value was used as an evaluation measure.

### Results

The accuracies of prediction were (1) 4.52±0.16% and (2) 16.52±0.30%. It was demonstrated that the accuracy of prediction can remarkably be improved with the genetic architecture division as compared with the cases without the genetic architecture division.

### Example 5

### Method

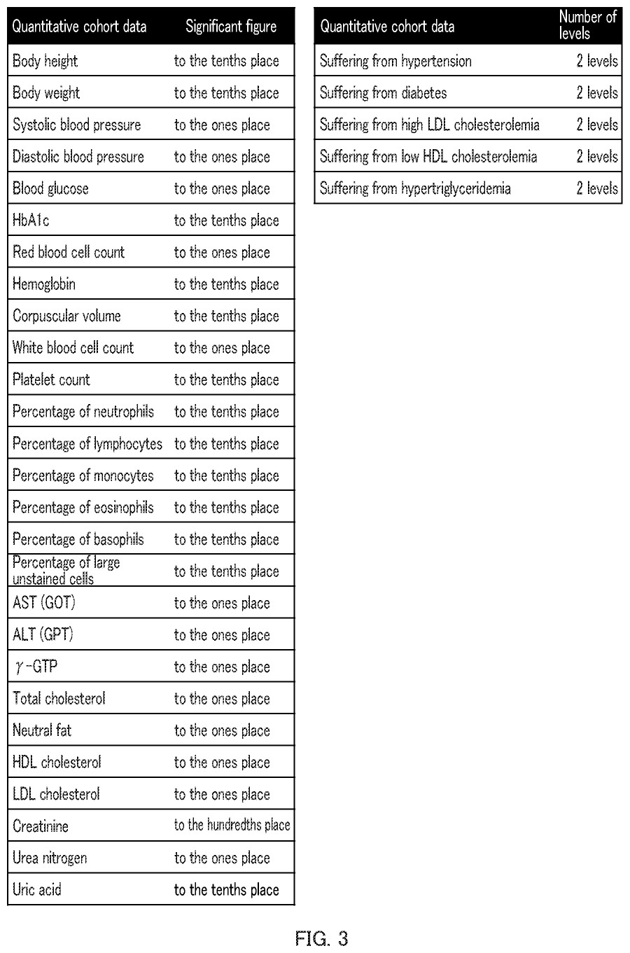

In this example, for 27 quantitative traits and 5 qualitative traits shown in FIG. 3, single nucleotide polymorphism data collected from 4,992 individuals from April 2015 to March 2016 by the Tohoku Medical Megabank Project were used and trait prediction models were made by the method of creating a trait prediction model of the present invention (using the aforementioned (9-3) with genetic architectures and gender/age information). The accuracy of prediction by the trait prediction model was evaluated for each of the cases where (1) only the single nucleotide polymorphism information was used and Qₑₛ=1 and Q_{RAF}=1 (without the genetic architecture division); (2) only the gender/age information was used; and (3) both the single nucleotide polymorphism information and the gender/age information were used and Qₑₛ=1 and Q_{RAF}=1 (without the genetic architecture division; the examples of the present invention), using a 2-fold cross validation method. The coefficient of determination R² (i.e., a squared correlation coefficient) between the measured value and the predicted value was used as an evaluation measure for the quantitative data and AUC was used for the qualitative data.

### Results

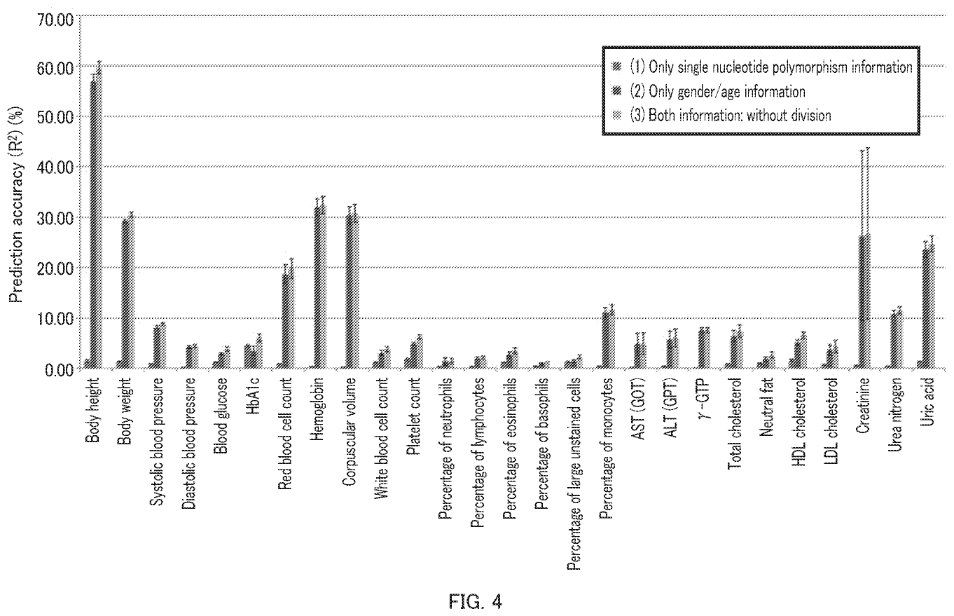

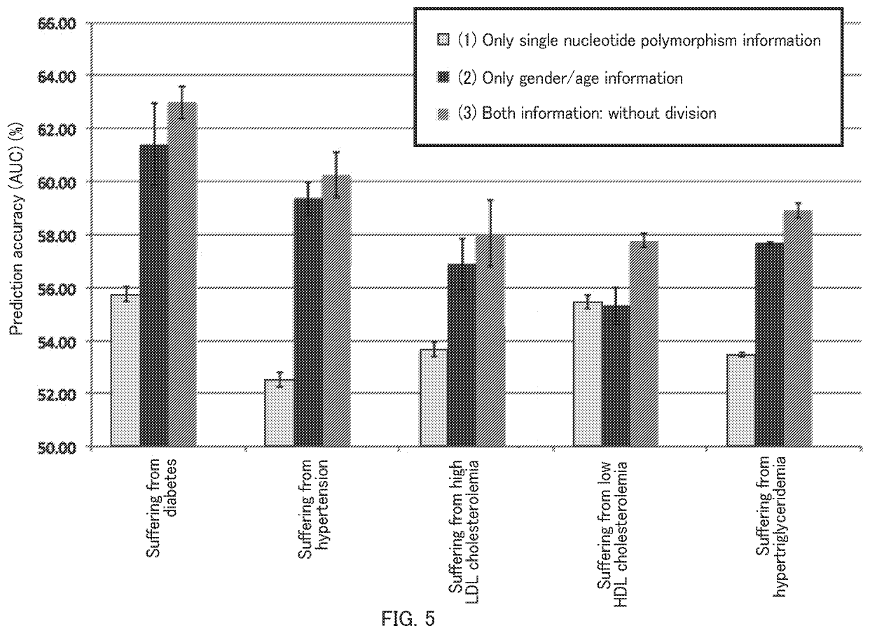

FIGS. 4 and 5 show the results of accuracy evaluation for the 27 quantitative traits and 5 qualitative traits, respectively. For all of the 27 quantitative traits and the 5 qualitative traits shown in FIGS. 4 and 5, it was demonstrated that the accuracies of prediction in (3) both the single nucleotide polymorphism information and the gender/age information were used and Qₑₛ=1 and Q_{RAF}=1 (without the genetic architecture division; the examples of the present invention) were higher than in (1) only the single nucleotide polymorphism information was used and Qₑₛ=1 and Q_{RAF}=1 (without the genetic architecture division); (2) only the gender/age information was used.

### Example 6

### Method

In order to show that the accuracy of trait prediction can be improved by using the gender/age information or both of the single nucleotide polymorphism information and the gender/age information when the training was performed using a sufficient amount of samples. For 27 quantitative traits and 5 qualitative traits shown in FIG. 3, single nucleotide polymorphism data collected from 4,992 individuals from April 2015 to March 2016 by the Tohoku Medical Megabank Project were used and trait prediction models were made by the method of creating a trait prediction model of the present invention (using the aforementioned (9-3) with genetic architectures and gender/age information). The accuracy of prediction by the trait prediction model was evaluated for each of the cases where (1) only the single nucleotide polymorphism information was used and Qₑₛ=1 and Q_{RAF}=1 (without the genetic architecture division); (2) only the gender/age information was used; (3) both the single nucleotide polymorphism information and the gender/age information were used and Qₑₛ=1 and Q_{RAF}=1 (without the genetic architecture division; the examples of the present invention); and (4) both the single nucleotide polymorphism information and the gender/age information were used and Qₑₛ=10 and Q_{RAF}=1 (with the genetic architecture division; the examples of the present invention), using a 2-fold cross validation method. The coefficient of determination R² (i.e., a squared correlation coefficient) between the measured value and the predicted value was used as an evaluation measure for the quantitative data and AUC was used for the qualitative data. Estimation of effect sizes and allele frequencies as well as estimation of linear mixed models were performed using a set of verification data. Prediction of contribution ratio by genetic factors and calculation of weights to single nucleotide polymorphisms were performed using a set of training data. The accuracy of prediction was verified using a set of verification data.

### Results

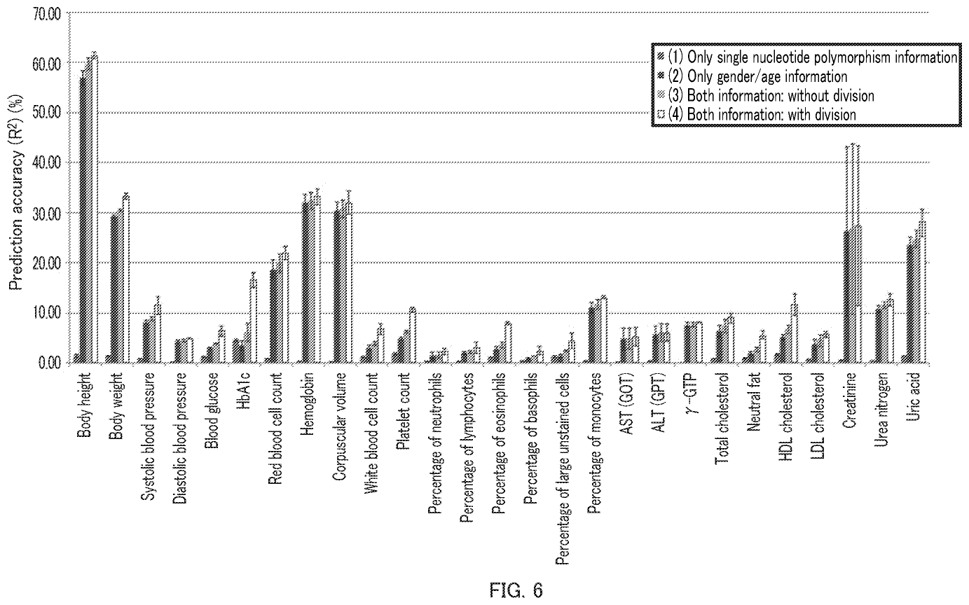

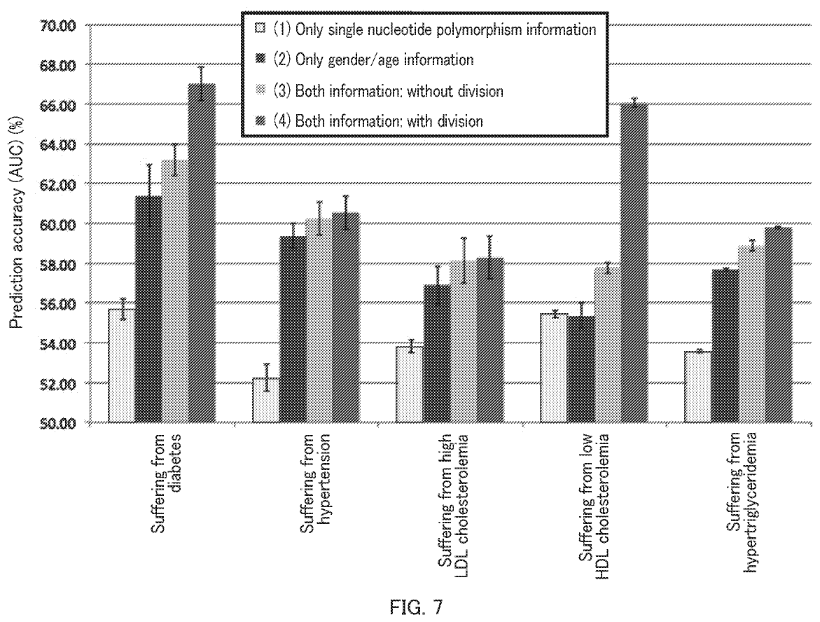

FIGS. 6 and 7 show the results of accuracy evaluation for the 27 quantitative traits and 5 qualitative traits, respectively. For all of the 27 quantitative traits and the 5 qualitative traits shown in FIGS. 6 and 7, it was demonstrated that the accuracies of prediction in (3) both the single nucleotide polymorphism information and the gender/age information were used and Qₑₛ=1 and Q_{RAF}=1 (without the genetic architecture division; the examples of the present invention) were higher than in (1) only the single nucleotide polymorphism information was used and Qₑₛ=1 and Q_{RAF}=1 (without the genetic architecture division); (2) only the gender/age information was used. For all traits, the accuracies of prediction in (4) both the single nucleotide polymorphism information and the gender/age information were used and Qₑₛ=10 and Q_{RAF}=1 (with the genetic architecture division; the examples of the present invention) were higher, when (3) both the single nucleotide polymorphism information and the gender/age information were used and Qₑₛ=1 and Q_{RAF}=1 (without the genetic architecture division; the examples of the present invention) and (4) both the single nucleotide polymorphism information and the gender/age information were used and Qₑₛ=10 and Q_{RAF}=1 (with the genetic architecture division; the examples of the present invention) were compared.

### Conclusion

As shown above, by using a trait prediction model created by a method of creating a trait prediction model of the present invention, traits can be predicted with a higher accuracy than with a conventional prediction method. Furthermore, it is possible to elucidate the genetic architecture of a trait by estimating the contribution ratio by the genetic architecture division method.

### Industrial Applicability

According to the present invention, it becomes possible to provide methods of creating a trait prediction model for predicting phenotypic traits from single nucleotide polymorphism data, and methods of predicting traits with which traits can be predicted with a high accuracy.

1. A computer-implemented method of creating a trait prediction model for predicting a phenotype of a multifactorial trait using data of gender, age and p single nucleotide polymorphisms linked to a trait for each of N individuals of an organism, the method comprising the steps of:
representing the single nucleotide polymorphisms as a matrix, wherein the matrix is defined by
$$W ( i , j ) = \{ \begin{matrix}\frac{- 2 {f}_{j}}{\sqrt{2 {f}_{j} ( 1 - {f}_{j} )}} & for “ AA ” \\ \frac{1 - 2 {f}_{j}}{\sqrt{2 {f}_{j} ( 1 - {f}_{j} )}} & for “ AB ” \\ \frac{2 - 2 {f}_{j}}{\sqrt{2 {f}_{j} ( 1 - {f}_{j} )}} & for “ BB ” ,\end{matrix}$$
wherein the j-th polymorphism of the i-th individual has two alleles; an individual with both alleles identical to a representative sequence is denoted as “AA”, an individual with only one allele identical to the representative sequence is denoted as “AB”, and an individual with both alleles not identical to the representative sequence is denoted as “BB”; the element in the i-th row and j-th column of the matrix W is denoted as W(i,j); the allele frequency of the j-th polymorphism is denoted as fⱼ; and the representative sequence is a sequence having nucleotides determined for respective polymorphisms;
representing the gender and/or age as a matrix X, wherein each row vector of the matrix X represents the gender/age information of the corresponding individual;
calculating a similarity matrix A using the represented matrix of the single nucleotide polymorphisms and a number of the single nucleotide polymorphisms as follows:
$${A}^{( i , j )} = \frac{1}{{p}^{( i , j )}} {W}^{( i , j )} {W}^{( i , j ) ′} ,$$
wherein A^{(i,j)} is a similarity matrix (N by N dimensions) for the category (i,j), p^{(i,j)} is the number of SNPs belonging to the category (i,j), W^{(i,j)} is a submatrix (N by p^{(i,j)} dimensions) obtained by taking a column vector or vectors of SNPs belonging to the category (i,j) from the matrix W, and W^{(i,j)}′ is a transpose of the submatrix W^{(i,j)}; and
applying the genomic similarity matrix and the matrix of the gender and/or age to a linear mixed model as follows:
y=μ1_{N}+Xβ+g+ε
g˜N(0,σ_{g}²A)
ε˜N(0,σₑ²I),
wherein y is a vector (N dimension) of traits, μ is a mean value of traits, 1_{N} is a column vector (N dimension) of which elements are all 1, X is a matrix containing the gender/age information, β is a weight for gender or age variables, g is a vector (N dimension) of genetic contributions to a trait, ε is a residual vector (N dimension), A is a genomic similarity matrix (N by N dimensions) when Qₑₛ=1 and Q_{RAF}=1, I is an identity matrix (N by N dimensions), N(0,σ_{g}²A) represents a multivariate normal distribution (with mean vector 0 and variance-covariance structure σ_{g}²A), and N(0,σₑ²I) represents a multivariate normal distribution (with mean vector 0 and variance-covariance structure σₑ²I); wherein
the trait is selected from the group consisting of the body height, body weight, systolic blood pressure, diastolic blood pressure, blood glucose, HbA1c, red blood cell number, hemoglobin, corpuscular volume, white blood cell number, platelet number, percentage of neutrophils, percentage of lymphocytes, percentage of monocytes, percentage of eosinophils, percentage of basophils, percentage of large unstained cells, AST (GOT), ALT (GPT), γ-GTP, total cholesterol, neutral fat, HDL cholesterol, LDL cholesterol, creatinine, urea nitrogen, uric acid, diabetes, hypertension, high LDL cholesterolemia, low HDL cholesterolemia, and hypertriglyceridemia.

2. The computer-implemented method of creating a trait prediction model for predicting a phenotype of a multifactorial trait using data of gender, age and p single nucleotide polymorphisms linked to a trait for each of N individuals of an organism, the method comprising the steps of:,
representing the single nucleotide polymorphisms as a matrix, wherein the matrix is defined by
$$W ( i , j ) = \{ \begin{matrix}\frac{- 2 {f}_{j}}{\sqrt{2 {f}_{j} ( 1 - {f}_{j} )}} & for “ AA ” \\ \frac{1 - 2 {f}_{j}}{\sqrt{2 {f}_{j} ( 1 - {f}_{j} )}} & for “ AB ” \\ \frac{2 - 2 {f}_{j}}{\sqrt{2 {f}_{j} ( 1 - {f}_{j} )}} & for “ BB ” ,\end{matrix}$$
wherein the j-th polymorphism of the i-th individual has two alleles; an individual with both alleles identical to a representative sequence is denoted as “AA”, an individual with only one allele identical to the representative sequence is denoted as “AB”, and an individual with both alleles not identical to the representative sequence is denoted as “BB”; the element in the i-th row and j-th column of the matrix W is denoted as W(i,j); the allele frequency of the j-th polymorphism is denoted as fⱼ; and the representative sequence is a sequence having nucleotides determined for respective polymorphisms;
classifying the single nucleotide polymorphisms into Qₑₛ-by-Q_{RAF} (0≤i≤Qₑₛ; 0≤j≤Q_{RAF}) categories based on their genetic architecture, wherein the genetic architecture is an effect size and/or an allele frequency;
representing the gender and/or age as a matrix X, wherein each row vector of the matrix X represents the gender/age information of the corresponding individual;
calculating a similarity matrix A using the represented matrix of the single nucleotide polymorphisms and a number of the single nucleotide polymorphisms as follows:
$${A}^{( i , j )} = \frac{1}{{p}^{( i , j )}} {W}^{( i , j )} {W}^{( i , j ) ′} ,$$
wherein A^{(i,j)} is a similarity matrix (N by N dimensions) for the category (i,j), p^{(i,j)} is the number of SNPs belonging to the category (i,j), W^{(i,j)} is a submatrix (N by p^{(i,j)} dimensions) obtained by taking a column vector or vectors of SNPs belonging to the category (i,j) from the matrix W, and W^{(i,j)}′ is a transpose of the submatrix W^{(i,j)}; and
applying the genomic similarity matrix and a parameter of the genetic architecture to a linear mixed model as follows:
$$y = {μ1}_{N} + X β + g + ɛ$$
$$g = {\sum }_{i , j} {g}^{( i , j )} \\ {g}^{( i , j )} ∼ N ( 0 , {σ}_{g}^{{2}^{( i , j )}} {A}^{( i , j )} ) \\ ɛ ∼ N ( 0 , {σ}_{e}^{2} I ) ,$$
where y is a vector (N dimension) of traits, μ is a mean value of traits, 1_{N} is a column vector (N dimension) of which elements are all 1, X is a matrix containing the gender/age information, β is a weight for gender or age variables, g is a vector (N dimension) of genetic contributions to a trait, ε is an residual vector (N dimension), g^{(i,j)} is a vector (N dimension) of contributions of SNPs belonging to the category (i,j) to a trait, A^{(i,j)} is a genomic similarity matrix (N by N dimensions) for the category (i,j), I is an identity matrix (N by N dimensions), N(0,σ_{g}^{2(i,j)}A^{(i,j)}) represents a multivariate normal distribution (with mean vector 0 and variance-covariance structure σ_{g}^{2(i,j)}A^{(i,j)}), and N(0,σₑ²I) represents a multivariate normal distribution (with mean vector 0 and variance-covariance structure σₑ²I); wherein
the trait is selected from the group consisting of the body height, body weight, systolic blood pressure, diastolic blood pressure, blood glucose, HbA1c, red blood cell number, hemoglobin, corpuscular volume, white blood cell number, platelet number, percentage of neutrophils, percentage of lymphocytes, percentage of monocytes, percentage of eosinophils, percentage of basophils, percentage of large unstained cells, AST (GOT), ALT (GPT), γ-GTP, total cholesterol, neutral fat, HDL cholesterol, LDL cholesterol, creatinine, urea nitrogen, uric acid, diabetes, hypertension, high LDL cholesterolemia, low HDL cholesterolemia, and hypertriglyceridemia.

3. A computer-implemented method of predicting a trait of an individual of an organism from a plurality of single nucleotide polymorphism data in the individual of the organism, comprising the steps of:
creating a trait prediction model using a set of training data according to the method of creating a trait prediction model according to claim 1;
determining a parameter and a hidden variable of a linear mixed model; and
applying the plurality of single nucleotide polymorphism data of the individual of the organism to the trait prediction model.

4. A non-transitory computer readable recording medium, comprising a program that causes the computer to execute the method according to claim 1.

5. A trait prediction system for predicting a trait of an individual of an organism from a plurality of single nucleotide polymorphism data, comprising:
(i) an input device for inputting a plurality of single nucleotide polymorphism data of the individual of the organism;
(ii) a computer that executes a program that causes the computer to execute the method according to claim 1 using the input data, and
(iii) an output device for outputting the result obtained in (ii).

6. A non-transitory computer readable recording medium, comprising a program that causes the computer to execute the method according to claim 2.

7. A trait prediction system for predicting a trait of an individual of an organism from a plurality of single nucleotide polymorphism data, comprising:
(i) an input device for inputting a plurality of single nucleotide polymorphism data of the individual of the organism;
(ii) a computer that executes a program that causes the computer to execute the method according to claim 2 using the input data, and
(iii) an output device for outputting the result obtained in (ii).

8. A non-transitory computer readable recording medium, comprising a program that causes the computer to execute the method according to claim 3.

9. A trait prediction system for predicting a trait of an individual of an organism from a plurality of single nucleotide polymorphism data, comprising:
(i) an input device for inputting a plurality of single nucleotide polymorphism data of the individual of the organism;
(ii) a computer that executes a program that causes the computer to execute the method according to claim 3 using the input data, and
(iii) an output device for outputting the result obtained in (ii).
